(19) United States
(12) Patent Application Publication
(10) Pub. No.: US 2019/0362807 A1
(43) Pub. Date: Nov. 28, 2019

(54) GENOMIC VARIANT RANKING SYSTEM FOR CLINICAL TRIAL MATCHING

(71) Applicant: KONINKLIJKE PHILIPS N.V., EINDHOVEN (NL)

(72) Inventor: Alexander Ryan Mankovich, Boston, MA (US)

(21) Appl. No.: 16/334,094

(22) PCT Filed: Sep. 28, 2017
(86) PCT No.: PCT/EP2017/074687

§ 371 Date: Mar. 18, 2019

Related U.S. Application Data
(60) Provisional application No. 62/401,319, filed on Sep. 29, 2016.

(51) Int. Cl. G16B 20/20; G16B 30/20; G16B 30/10

(52) U.S. Cl. CPC G16B 20/20; G16B 30/10; G16B 30/20

(57) ABSTRACT

A genetic sequencer (10) generates DNA reads (16) from a tissue sample of a current patient. The DNA reads are aligned (18) with a reference DNA sequence (20) to generate a DNA sequence (22) of the current patient. Variant calling (24) is performed to generate a list of genetic variants (26) contained in the DNA sequence of the current patient. Occurrences are determined of genetic variants in one or more reference databases (44) storing genetic variants of medical patients. It is determined whether genetic variants of the list of genetic variants are synonymous. Scores are assigned for genetic variants based at least on measures of correlation of the genetic variants with disease. A ranked list (32) of top-scoring genetic variants is generated based on the assigned scores, and the ranked list is displayed on a display (36).

### FIELD

The following relates generally to the genetic sequencing arts, medical diagnosis and treatment arts, and related arts.

### BACKGROUND

Whole genome deoxyribonucleic acid (DNA) sequencing is becoming increasingly affordable in the clinical setting, such that it is becoming feasible to obtain a whole genome DNA sequence for an oncology patient (or, more generally, for a patient having a disease that may correlate with a genetic variant or some combination of genetic variants). Such sequencing is typically performed on cancer tissue of a patient with late stage cancer for whom more conventional therapies have been ineffective. In other clinical tasks, normal tissue may be sampled. For example, numerous diseases other than cancer have genetic variant(s) that are correlative with the disease, e.g. the genetic variant may predispose the person to a higher likelihood of the disease. The whole genome sequence is processed to generate reads which are aligned with a reference sequence to produce the genomic sequence. Variant calling is performed to identify nucleotides that differ from the reference sequence, and these called variants are stored, typically in a Variant Call Format (VCF) file that lists, for each variant, the chromosome, position in the chromosome, a variant label or identifier (if known), the expected reference nucleotide, the actual nucleotide in the patient's tissue, and possibly other information such as a read quality metric. The list of variants may then be mined to identify clinically relevant information.

The following discloses a new and improved systems and methods.

### SUMMARY

In one disclosed aspect, a non-transitory storage medium stores instructions readable and executable by an electronic processor to perform a genetic variant ranking method comprising: assigning dataset detection scores for genetic variants of a list of genetic variants of a current patient's deoxyribonucleic acid (DNA) sequence wherein the dataset detection scores are measures of occurrences of the genetic variants in one or more reference databases storing genetic variants of medical patients; assigning functional scores for genetic variants of the list of genetic variants wherein the functional scores are measures of impact of the genetic variants on gene transcription; assigning disease correlation scores for genetic variants of the list of genetic variants wherein the disease scores are measures of correlation of the genetic variants with disease; assigning transcriptomics scores for genetic variants of the list of genetic variants wherein the transcriptomics scores are measures of expression of the genetic variants in at least one of ribonucleic acid (RNA) transcript data and microarray data for the current patient; generating a ranked list of top-scoring genetic variants of the list of genetic variants based on the dataset detection, functional, disease, and transcriptomics scores; and displaying the ranked list of top-scoring genetic variants on a display device operatively connected with the electronic processor.

In another disclosed aspect, a genetic sequencing and processing system is disclosed. A genetic sequencer is configured to generate DNA reads from a tissue sample of a current patient. The system further includes an electronic processor, a display, and a non-transitory storage medium storing instructions readable and executable by the electronic processor to: align the DNA reads with a reference DNA sequence to generate a DNA sequence of the current patient; perform variant calling to generate a list of genetic variants contained in the DNA sequence of the current patient; determine occurrences of genetic variants of the list of genetic variants in one or more reference databases storing genetic variants of medical patients and discard any genetic variants for which the determined occurrences do not satisfy a threshold occurrence level; determine whether genetic variants of the list of genetic variants are synonymous and discard any genetic variants which are determined to be synonymous; assign scores for genetic variants of the list of genetic variants that are not discarded wherein the scores are based at least on measures of correlation of the genetic variants with disease; generate a ranked list of top-scoring genetic variants of the list of genetic variants that are not discarded based on the assigned scores; and display the ranked list of top-scoring genetic variants on the display.

In another disclosed aspect, a genetic variant ranking method comprises: filtering a list of genetic variants of a current patient's DNA sequence to discard genetic variants whose occurrences in one or more reference databases storing genetic variants of medical patients does not meet a threshold occurrence level; assigning disease correlation scores for genetic variants of the list of genetic variants wherein the disease scores are measures of correlation of the genetic variants with disease; assigning transcriptomics scores for genetic variants of the list of genetic variants wherein the transcriptomics scores are measures of expression of the genetic variants in at least one of ribonucleic acid (RNA) transcript data and microarray data for the current patient; generating a ranked list of top-scoring genetic variants of the list of genetic variants based on at least the disease correlation and transcriptomics scores; and displaying the ranked list of top-scoring genetic variants on a display device. The filtering, assigning of disease and transcriptomics scores, and generating of the ranked list are suitably performed by an electronic processor.

One advantage resides in providing an improved genetic sequencing and processing system with improved clinical usefulness.

Another advantage resides in providing more targeted genetic analysis for a patient in a clinical setting, thereby facilitating more efficient use of the provided genetic information.

Another advantage resides in providing an improved genetic sequencing and processing system with greater computational efficiency.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a diagnostic tool employing genomic sequencing to match a patient with clinical treatments or clinical trials or the like.

FIG. 2 diagrammatically illustrates a graphical transcriptomics representation.

FIG. 3 shows a table of two illustrative scored genetic variants.

### DETAILED DESCRIPTION

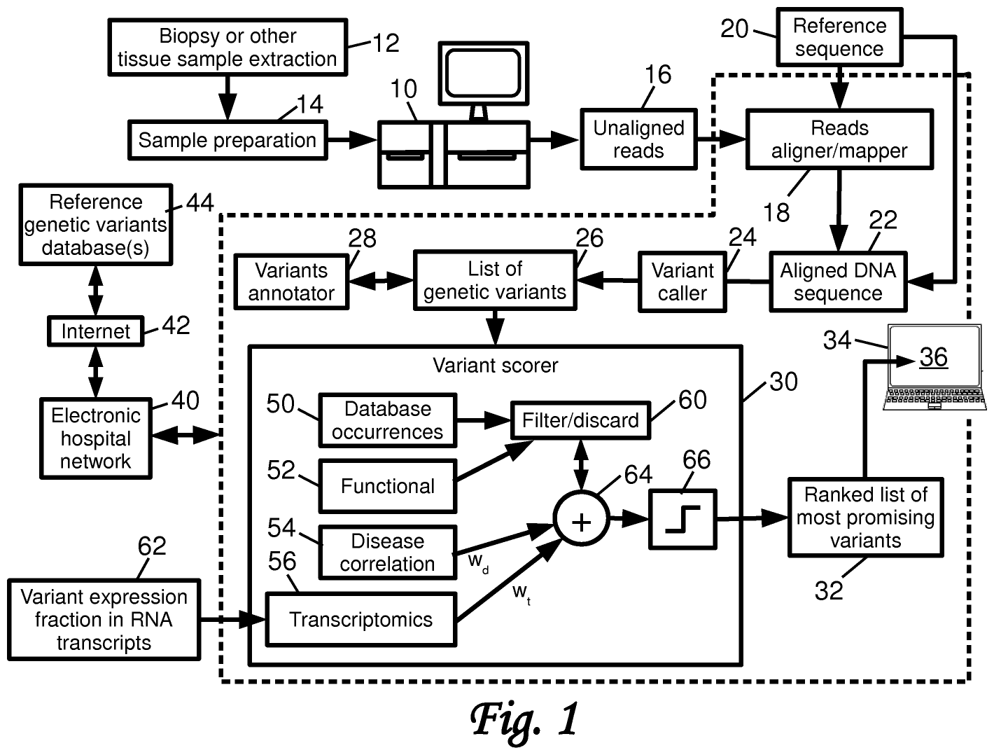

With reference to FIG. 1, an illustrative genetic sequencing and processing system includes a genetic sequencer 10. To use the sequence 10, a clinician draws a tissue sample from a current patient, e.g. via a biopsy procedure or other tissue extraction procedure 12 that draws a tissue sample from a malignant tumor. Various sample preparation 14 is performed as is known in the art, e.g. wet lab procedures to extract purified deoxyribonucleic acid (DNA) from the sample, perform end repair/modification, polymerase chain reaction (PCR) amplification, and so forth. The resulting DNA sample is loaded into the genetic sequencer 10, typically using a sample cartridge designed for this purpose. The genetic sequencer 10 operates to generate unaligned DNA sequence fragment reads, that is, data representations of base sequences of DNA fragments, preferably with read confidence (i.e. “quality”) scores for the bases of the sequence. The DNA fragment reads 16 may, for example, be stored in the commercially common FASTQ format. By way of non-limiting illustrative example, the genetic sequencer 10 may, for example, comprise an Illumina™, PacBio™, Ion Torrent™, Nanopores™, ABI-SOLiD™, or other commercially available genetic sequencer. The sample preparation 14 is typically tailored to the chosen genetic sequencer 10 and is performed in accordance with procedures promulgated by the sequencer manufacturer and, in some instances, using proprietary chemicals provided by the sequencer manufacturer. Depending upon the choice of processing, the DNA sample and consequently the reads 16 may be limited to a particular type or selection of DNA, e.g. selective PCR may be used to selectively amplify only certain DNA portions. As another example, in whole exome sequencing (WES), only the expressed genes (i.e., protein-encoding exons) are sequenced, by using known target enrichment processing to isolate the exons (or only selected exons). If the DNA isolation/amplification processing is not selective, then all DNA material is isolated and amplified, thus providing for whole genome sequencing (WGS).

The unaligned reads 16 are aligned or mapped by a reads aligner/mapper 18 to a reference sequence 20 to generate an aligned DNA sequence, which may be a WES, WGS, or the like depending upon the preparatory tissue sample processing 14. By way of non-limiting illustrative example, the reads aligner/mapper 18 may for example comprise a Burrows-Wheeler Alignment (BWA) tool for performing short read alignment followed by a processing by the SAMtools suite to align longer sequences. The resulting aligned sequence 22 may, for example, be stored in a commercially standard Sequence Alignment/Map (SAM) or Binary Alignment Map (BAM) format.

A variant caller 24 employs suitable approaches for identifying genetic variants in the aligned DNA sequence 22 of the current patient. The genetic variants may be single nucleotide substitution variants, sometimes referred to as single nucleotide polymorphism (SNP) or single nucleotide variant (SNV) variants; base modification variants (e.g. methylation), an “extra” inserted base or a missing, i.e. “deleted” base, commonly referred to collectively as indels, copy number variations (CNVs), or so forth. In a suitable approach, the variant caller 24 calls genetic variants contained in the DNA sequence 22 of the current patient as compared with the reference DNA sequence 20. To account for low read coverage and other complications, the variant caller 24 may employ probabilistic or statistical methods for identifying genetic variants. Numerous research-grade and commercial variant calling tools are known and may be employed (optionally in various combinations) to implement the variant caller 24. The resulting list of genetic variants 26 of the current patient's DNA sequence 22 is suitably stored in a standard variant calls file (VCF) format. In one standard VCF format, each variant is stored as <#Chrome Pos ID Ref Alt Qual> where “#Chrome” identifies the chromosome containing the variant, “Pos” identifies the position of the variant on that chromosome, “ID” is an identification of the variant (optional, e.g. provided by a variants annotator 28), “Ref” identifies the reference base (from the reference sequence 20, assuming a simple SNV or SNP variant), “Alt” stores the actual (substitute) base in the current patient's DNA sequence 22 (again assuming a simple SNV or SNP variant), and “Qual” is a confidence level or quality metric for the variant. Fewer, additional, or other fields may be provided.

In a variant embodiment, which may be suitable in oncology tasks, the biopsy or other tissue extraction 12 is performed to obtain two tissue samples: a cancer tissue sample (e.g. from a malignant tumor) and a non-cancer tissue sample (e.g. from tissue not containing metastasized cancer cells). Both the cancer and non-cancer tissue samples are drawn from the same current patient. Both samples are processed 14 in the same way, and the genetic sequencer 10 generates DNA reads from the cancer tissue sample of the current patient and also from the non-cancer tissue sample of the current patient. The DNA reads of the non-cancer tissue sample are aligned by the aligner 18 with the reference DNA sequence 20 to generate a non-cancer DNA sequence of the current patient. Similarly, the DNA reads of the cancer tissue sample are aligned by the aligner 18 with the reference DNA sequence 20 (or, alternatively, are aligned with the previously aligned non-cancer DNA sequence of the current patient, or some combination of these alignments may be performed) to generate a cancer DNA sequence of the current patient. The variant caller 24 then generates the list of genetic variants 26 for the current patient contained in the cancer DNA sequence of the current patient as compared with the non-cancer DNA sequence of the current patient. This approach may have an advantage insofar as the called variants will be strongly attributable to the cancer. (However, it should be noted that in other embodiments, an oncology task may be performed using only genetic sequencing of cancer tissue, with variants being identified by comparison with a reference DNA sequence rather than to the patient's own normal tissue).

The annotator 28 may take various forms, e.g. some non-limiting examples of available tools for annotating somatic mutations in the VCF file 26 include: SIFT, Polyphen-2, Mutation Assessor, Condel, FATHMM, CHASM, and transFIC. Each tool employs a tool-specific method for predicting the functional impact of non-synonymous (i.e. amino-acid changing) variants. Certain variants, for instance, may alter the amino acid, but not impact the overall three-dimensional (3D) structure of the protein and therefore not impact its function in the cell. While a variant may impact cellular function, it is not always the case that there is a therapy targeting such dysfunction.

The resulting list of genetic variants 26 has numerous potential clinical benefits. The genetic variants can be employed for clinical trial matching to identify a possible avenue for new or alternative treatment of a patient with late-stage cancer or another disease correlative with genetic variant(s). However, clinical trial matching can be a computationally complex process that can take a significant amount of time, especially with the high number of variants detected in a WES or WGS. This is problematic both in terms of occupying valuable clinician time, and insofar as treatment of late-stage cancer or other debilitating or life-threatening diseases is a time-critical task. A WES or WGS may have millions of genomic variants, presenting a difficult problem for clinicians to efficiently identify the most clinically useful genetic variants.

To address these difficulties, a variant scorer 30 operates to generate a ranked list of the most promising variants 32. The variant scorer 30 identifies whether the variant exists in other datasets (or, conversely, is so rare that finding a matching clinical trial is unlikely). Variants are also scored on other factors such as functional impact (e.g. does it affect transcription of a gene?) and disease correlation. By selecting the most important (i.e. highest-scoring) variants 32 based on existential, functional, and disease-related annotations, the complexity of clinical trial matching can be drastically reduced.

With continuing reference to FIG. 1, the various processing components, e.g. the reads aligner 18, variant caller 24, variant annotator 28, and variant scorer 30, are suitably implemented on a computer or other electronic processor 34 which reads and executes instructions stored on a non-transitory storage medium, which instructions when executed by the electronic processor 34 implement the various computational components, e.g. the reads aligner 18, variant caller 24, variant annotator 28, and variant scorer 30. While the illustrative electronic processor 34 is a desktop computer, it may alternatively or additionally comprise a server computer, a cluster of server computers, a distributed computing resource in which electronic processors are operatively combined on an ad hoc basis (e.g. a cloud computing resource), an electronic processor of the genetic sequencer 10, and/or so forth. The non-transitory storage medium storing the instructions which are read and executed by the electronic processor 34 may, for example, comprise one or more of: a hard disk drive or other magnetic storage medium; a flash memory, solid state drive (SSD), or other electronic storage medium; an optical disk or other optical storage medium; and/or so forth. Furthermore, the electronic processor 34 includes or is operatively connected with a display 36 on which the ranked list of highest-scoring genetic variants 32 may be displayed. The computer or other electronic processor 34 is also operatively connected with an electronic hospital network 40 or the like, and via such network 40 may be connected with the Internet 42 and/or one or more regional or global reference genetic variants databases 44, such as by way of non-limiting illustration the Beacon network (https://beacon-network.org).

With continuing reference to FIG. 1, some illustrative embodiments of the variant scorer 30 are described. The illustrative scorer operates on the basis of four filtering or scoring factors: filtering or scoring on the basis of database occurrences 50 of the genetic variant; filtering or scoring on the basis of functional assessment 52 of the variant; filtering or scoring on the basis of disease correlation 54; and filtering or scoring on the basis of transcriptomic analysis 56 of the genetic variant. These are described below in turn.

The dataset detection 50 is based on the recognition that a genetic variant that has not been identified elsewhere is not likely to be clinically useful. Thus, the dataset detection 50 is useful in variant prioritization in regards to treatment and clinical trial matching. If a genetic variant does not exist (or is very rare) in other patients it is very unlikely a clinical trial will be designed specifically targeting that variant. The dataset detection 50 annotates variants with results from querying one or more external reference genetic variants databases 44 (such as the Beacon network, https://beacon-network.org) and/or one or more internal reference genetic variants databases (such as a hospital information technology system, an Electronic Medical Record, or so forth). In one suitable design, the dataset detection 50 returns a value of ‘true’ if the variant exists in one of these reference databases, or returns ‘false’ otherwise. (In other contemplated embodiments, there may be some minimum threshold for returning ‘true’, e.g. the variant must have occurred in at least N other patients to be ‘true’ where N may be greater than one). The reference patients databases should be sufficiently large enough (preferably on the order of hundreds of thousands or millions of patients) to be confident in the result. Other scoring frameworks may be used, e.g. the dataset detection 50 may return a value of 100 or 0 for ‘detected’ or ‘not detected’, respectively. This category is preferably heavily weighted in computing a composite score for the variant, or even more preferably (as in the illustrative embodiment of FIG. 1) may be used as input to a filter 60 that discards any variant not meeting filter criteria, i.e. any variant that does not return ‘true’ indicating it exists in the reference database(s) may be discarded by the filter 60.

The functional analysis 52 provides one or more annotations (which can optionally range in the hundreds) indicating the functional significance of a genetic variant. The functional analysis 52 determines whether the genetic variant is synonymous or non-synonymous. A synonymous variant is one which does not impact the expression of the gene containing the variant. More particularly, if a SNP does not change the transcribed amino acid produced by the base triplet containing the SNP, then this is a synonymous variant. On the other hand, if the SNP does change the transcribed amino acid produced by the base triplet containing the SNP, then this is a non-synonymous variant. A synonymous variant has no functional effect on the gene and hence is unlikely to be of clinical importance; whereas, a non-synonymous variant does have a functional effect on the gene and may therefore be more likely to have deleterious clinical effect. In one embodiment of the functional analysis 52, only variants which are identified as non-synonymous are considered, and only annotations indicating deleteriousness/pathogenicity are weighed (such as SIFT, Polyphen-2, Mutation Assessor, Condel, FATHMM, CHASM, and transFlC cancer-impact tools). The value of each weighed annotation is a value of 1 or 0 (or a scaled value between 1 and 0 for annotations with numeric values), depending on whether the conclusion is deleterious/pathogenic or not. If several functional analysis tools are available, then the overall functional analysis 52 may return the average of the values output by the several tools. These values are only considered for annotations that exist in each variant. In the illustrative embodiment, the functional analysis 52 is again used as an input to the filter 60 so as to discard synonymous variants. In an alternative embodiment, if there are several functional analysis tools such that the final output is not definitively synonymous or definitively non-synonymous, the output may be used as a score that is incorporated into the composite score.

The disease correlation 54 is useful for identifying clinical trials or therapies targeting that specific disease. Supplied with the disease indication of the current patient (such as a cancer or other disease diagnosis of the current patient), and the disease (or diseases) associated with the genetic variant (for example, obtained from a database such as ClinVar, or the Jackson Laboratory's Clinical Knowledgebase), the variant can be scored as to its disease correlation. In one approach, the disease correlation score is computed as follows: if the variant is correlated with the disease of the current patient (e.g. correlated with the same type of cancer afflicting the current patient) then the disease correlation score is set to its highest value (e.g. 1 in an illustrative example). If the variant is not associated with any disease (e.g. does not correlate with any type of cancer or other disease), then the disease correlation score is set to its lowest value (e.g. 0 in an illustrative example). Finally, if the variant is not associated with the disease of the current patient but is associated with some other disease (e.g. does not correlate with the cancer disease afflicting the current patient but does correlate with some other type of cancer, or with some type of non-cancer disease), then the disease correlation score is set to a value between the highest and lowest values (e.g. 0.5 in an illustrative example).

The transcriptomics analysis 56 can provide additional insight about a variant. Some functional prediction tools (e.g. Ensembl Variant Effect Predictor) supply all transcripts associated with a particular variant. However, not all of these transcripts are actively expressed. Cross-referencing transcriptomic data enables the system to assign higher priority to a variant if the transcript annotations matching the variant are being actively expressed.

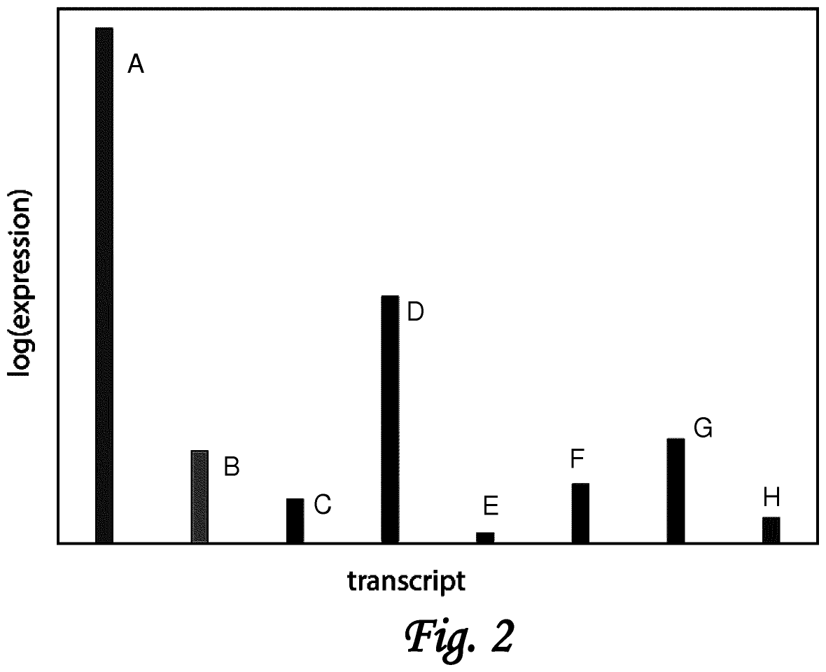

With brief reference to FIG. 2, an example of this is shown for nine genetic variants labelled “A”, “B”, . . . , “H”. Variants “A” and “D” are most strongly expressed, and hence are scored highest as to the transcriptomics analysis 56.

In some embodiments a transcriptomics score for a genetic variant of the list of genetic variants 26 is assigned based on information acquired for the current patient, rather than relying upon a generic database. Thus, the transcriptomics scores may be measures of expression of the genetic variants in at least one of ribonucleic acid (RNA) transcript data and microarray data for the current patient. For example, a variant a transcriptomics score may be assigned which is indicative of the fraction 62 of RNA transcripts of a gene to which the variant belongs that express the variant.

The variant scorer integrates the information from the analyses 50, 52, 54, 56 to generate a final score for the genetic variant. For analyses 50, 52 which operate as inputs to the filter 60, this integration entails discarding any variants that do not meet some criterion defined by the analysis (e.g., discarding any variant that does not meet a threshold occurrence level in the case of the dataset detection 50, or discarding any variant which is determined by the functional analysis 52 to be synonymous). For those analyses used in scoring, each variant that is not discarded by the filtering 60 is scored as a weighted sum 64 of the individual measures or scores output by the analysis 54, 56, e.g. in FIG. 1 the disease correlation score is weighted by a weight w_{d} while the transcriptomics score is weighted by a weight wₜ.

It should be noted that in other contemplated embodiments the database occurrences analysis 50 and/or the functional analysis 52 may be treated as scores rather than filters, and may then be included in the weighted sum 64 with suitable weights. For example, as already noted if the functional analysis 52 employs a plurality of tools such that the final output is not definitively either synonymous or non-synonymous, then it may be more suitable to treat the functional output as a scoring component fed into the weighted sum 64. Likewise, if the reference patient databases searched in the database occurrences analysis 50 are large enough, it may be useful to define the output of the database occurrences analysis 50 as something other than a binary ‘true’ or ‘false’ value, which may then be more effectively handled as a scoring component.

As another contemplated modification, the filtering 60 may also filter variants by discarding any genetic variant for which a confidence metric of the genetic variant is below a threshold. This may, for example, leverage the quality metric assigned to each base in the FASTQ format, so that variants of low confidence are discarded.

The variants are then ranked by the composite scores output by the weighted sum 64 and the top scoring variants from the ranked list 32 of top-scoring genetic variants. In this regard, any variants that are discarded by the filter 60 are automatically ranked at the bottom of the ranking and cannot be included in the ranked list 32. In the illustrative embodiment, a threshold 66 is employed, i.e. only (non-discarded) variant whose summed score is above the threshold 66 are included in the ranked list 32. In another approach, the ranked list 32 may be a “top K” list, i.e. the K variants with highest scores may be included.

The display of the ranked list 32 may include only identification of the top-scoring variants. Alternatively, the display may include displaying the transcriptomics scores assigned to the variants of the ranked list by the transcriptomics analysis 56, which can be useful information for the clinician in assessing the clinical importance of the variants. Similarly, other scores (e.g. the disease correlation score) or annotations relating to scores (e.g. identification of the disease(s) with which a variant is correlated) may be displayed.

A more detailed illustrative example follows. A biopsy 12 is sequenced according to an approved laboratory protocol (for example, whole exome sequencing). The sequencing data is processed by a variant calling pipeline 24 (process wherein genomic variants are detected and output in a standard format). Variants are filtered for quality, depth, and other standard metrics. Then, variants are given functional/clinical annotations. The highest priority variants will automatically be those with matching (non-)FDA approved therapies either within or outside the patient's primary disease indication. There are relatively few of these variants, and if none appear in the sample the clinician is then faced with identifying the relative importance of the remaining bulk of variants. This is where the variant scorer 30 is suitably employed and, according to the categorical weights provided, ranks the remaining variants by prioritizing according to the analyses 50, 52, 54, 56. Due to the costs and complexity of variant-based clinical trial matching, the clinician may only want to select the most likely (i.e., highest ranking) matches 32 as candidates.

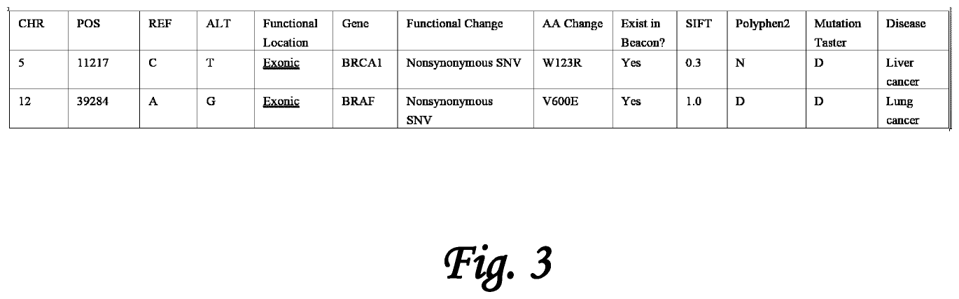

With reference to FIG. 3, in an example case, the variants shown in the table of FIG. 3 (and many like them) may appear in the results. However, it is difficult to automatically prioritize one over the other. In FIG. 3, the two variants are in well-known cancer genes, with functionally impactful alterations (non-synonymous), and have at least one report of deleteriousness from an annotation. For the first variant, it would be scored as follows: SIFT—0.3; Polyphen—0; MutationTaster—1. The total score of this variant is 100 (because this variant was detected elsewhere)+0.43 (average of functional scores)+0.5 (outside disease indication)=100.93. The total score of the second variant is 100 (detected elsewhere)+1.0 (average of functional scores)+1.0 (within disease indication)=102.00. With this scoring system, the genetic variant on chromosome 12 shown in FIG. 3 would be ranked higher than the genetic variant on chromosome 5 shown in FIG. 3.

If the clinician also has access to transcriptomic (i.e. expression) data 62 from the sample, in addition to the variant information. In this case, a final check may be run for whether the transcripts containing the detected variants are actually being expressed, and removes variants entirely when they are not (in this illustrative example, using the transcriptomics analysis 56 as part of the filter 60).

The illustrative examples have been directed to cancer. However, more generally, the disclosed genetic variant ranking approaches may be applied for identifying genetic variants relevant for diseases other than cancer diseases, e.g. for detecting congenital genetic disorders using germline testing or so forth.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A non-transitory storage medium storing instructions readable and executable by an electronic processor to perform a genetic variant ranking method comprising:
assigning dataset detection scores for genetic variants of a list of genetic variants of a current patient's deoxyribonucleic acid (DNA) sequence wherein the dataset detection scores are measures of occurrences of the genetic variants in one or more reference databases storing genetic variants of medical patients;
assigning functional scores for genetic variants of the list of genetic variants wherein the functional scores are measures of impact of the genetic variants on gene transcription;
assigning disease correlation scores for genetic variants of the list of genetic variants wherein the disease scores are measures of correlation of the genetic variants with disease;
assigning transcriptomics scores for genetic variants of the list of genetic variants wherein the transcriptomics scores are measures of expression of the genetic variants in at least one of ribonucleic acid (RNA) transcript data and microarray data for the current patient;
generating a ranked list of top-scoring genetic variants of the list of genetic variants based on the dataset detection, functional, disease, and transcriptomics scores; and
displaying the ranked list of top-scoring genetic variants on a display device operatively connected with the electronic processor.

2. The non-transitory storage medium of claim 1 wherein the generating of the ranked list includes:
discarding any genetic variants of the list of genetic variants for which the data detection score of the genetic variant indicates the genetic variant is not found in the one or more reference databases at above a threshold occurrence level.

3. The non-transitory storage medium of claim 1 wherein the generating of the ranked list includes:
discarding any genetic variants of the list of genetic variants for which the functional score of the genetic variant indicates the genetic variant is synonymous.

4. The non-transitory storage medium of claim 1, wherein the assigning of disease correlation scores includes:
assigning a variant with a lowest value if the variant is not correlated with any disease;
assigning a variant with a highest value if the variant is correlated with a disease of the current patient; and
assigning a variant with a score between the lowest score and the highest score if the variant is correlated with a disease that is not the disease of the patient.

5. The non-transitory storage medium of claim 1 wherein the assigning of transcriptomics scores includes:
assigning a variant a transcriptomics score indicative of a fraction of RNA transcripts of a gene to which the variant belongs that express the variant.

6. The non-transitory storage medium of claim 1 wherein the generating of the ranked list includes:
discarding any genetic variant of the list of genetic variants that meets a removal criterion wherein the removal criterion includes at least discarding any genetic variant for which a confidence metric of the genetic variant is below a threshold; and
assigning a combined score for each variant of the list of variants that is not discarded wherein the combined score comprises a weighted sum of at least two of the dataset detection score, functional score, disease correlation score, and transcriptomics score.

7. The non-transitory storage medium of claim 1 wherein:
the generating of the ranked list includes assigning combined scores for the variants comprising weighted sums of at least the disease correlation score and the transcriptomics score; and
the displaying of the ranked list of top-scoring genetic variants includes displaying the transcriptomics scores assigned to the variants of the ranked list.

8. A genetic sequencing and processing system comprising:
a genetic sequencer configured to generate deoxyribonucleic acid (DNA) reads and ribonucleic acid (RNA) transcript data from a tissue sample of a current patient;
an electronic processor;
a display; and
a non-transitory storage medium storing instructions readable and executable by the electronic processor to:
align the DNA reads with a reference DNA sequence to generate a DNA sequence of the current patient;
perform variant calling to generate a list of genetic variants contained in the DNA sequence of the current patient;
determine occurrences of genetic variants of the list of genetic variants in one or more reference databases storing genetic variants of medical patients and discard any genetic variants for which the determined occurrences do not satisfy a threshold occurrence level;
determine whether genetic variants of the list of genetic variants are synonymous and discard any genetic variants which are determined to be synonymous;
determine measures of expression of the genetic variants in the RNA transcript data and/or in received microarray data for the current patient;
assign scores for genetic variants of the list of genetic variants that are not discarded wherein the scores are based at least on measures of correlation of the genetic variants with disease and based on the measures of expression;
generate a ranked list of top-scoring genetic variants of the list of genetic variants that are not discarded based on the assigned scores; and
display the ranked list of top-scoring genetic variants on the display.

9. The genetic sequencing and processing system of claim 8 wherein the measures of correlation of the genetic variants with disease include:
assigning to a variant a lowest measure of correlation value if the variant is not correlated with any disease;
assigning a variant with a highest measure of correlation value if the variant is correlated with a cancer disease of the current patient; and
assigning a variant with a measure of correlation value between the lowest measure of correlation value and the highest measure of correlation value if the variant is correlated with a cancer disease that is not the cancer disease of the patient.

10. (canceled)

11. (canceled)

12. The genetic sequencing and processing system of claim 8 wherein the displaying of the ranked list of top-scoring genetic variants on the display includes displaying the measures of expression determined for the variants of the ranked list.

13. The genetic sequencing and processing system of claim 8 wherein the genetic sequencer is configured to generate DNA reads from a cancer tissue sample of the current patient and a non-cancer tissue sample of the current patient, and the non-transitory storage medium stores instructions readable and executable by the electronic processor to:
align the DNA reads of the non-cancer tissue sample with the reference DNA sequence to generate a non-cancer DNA sequence of the current patient, and align the DNA reads of the cancer tissue sample with at least one of the reference DNA sequence and the non-cancer DNA sequence of the current patient to generate a cancer DNA sequence of the current patient;
perform the variant calling to generate the list of genetic variants for the current patient contained in the cancer DNA sequence of the current patient as compared with the non-cancer DNA sequence of the current patient.

14. The genetic sequencing and processing system of claim 8 wherein the variant calling to generate the list of genetic variants contained in the DNA sequence of the current patient as compared with the reference DNA sequence.

15. The genetic sequencing and processing system of claim 8 wherein the instructions stored by the non-transitory storage medium are further readable and executable by the electronic processor to:
discard any genetic variant of the list of genetic variants for which a confidence metric of the genetic variant is below a threshold.

16. A genetic variant ranking method comprising:
filtering a list of genetic variants of a current patient's deoxyribonucleic acid (DNA) sequence to discard genetic variants whose occurrences in one or more reference databases storing genetic variants of medical patients does not meet a threshold occurrence level;
assigning disease correlation scores for genetic variants of the list of genetic variants wherein the disease scores are measures of correlation of the genetic variants with disease;
assigning transcriptomics scores for genetic variants of the list of genetic variants wherein the transcriptomics scores are measures of expression of the genetic variants in at least one of ribonucleic acid (RNA) transcript data and microarray data for the current patient;
generating a ranked list of top-scoring genetic variants of the list of genetic variants based on at least the disease correlation and transcriptomics scores; and
displaying the ranked list of top-scoring genetic variants on a display device;
wherein the filtering, assigning of disease and transcriptomics scores, and generating of the ranked list are performed by an electronic processor.

17. The variant ranking method of claim 16 wherein the filtering further discards genetic variants which are synonymous.

18. The variant ranking method of claim 16 wherein the assigning of disease correlation scores includes:
assigning a variant with a lowest disease score value if the variant is not correlated with disease;
assigning a variant with a highest disease score value if the variant is correlated with a disease of the current patient; and
assigning a variant with a disease score value between the lowest disease score value and the highest disease score value if the variant is correlated with a disease that is not a disease of the current patient.

19. The variant ranking method of claim 16 wherein the assigning of transcriptomics scores includes:
assigning a variant a transcriptomics score indicative of a fraction of RNA transcripts of a gene to which the variant belongs that express the variant.

20. The variant ranking method of claim 16 wherein the assigning of transcriptomics scores includes:
assigning a variant a transcriptomics score indicative of expression level of a gene to which the variant belongs that express the variant compared with expression level of the gene that does not express the variant.
